(19) United States
(12) Patent Application Publication
(10) Pub. No.: US 2005/0182372 A1
(43) Pub. Date: Aug. 18, 2005

(54) Wearable and regenerative flatus and genitalia odor removal apparatus, method of making same and method of removing such odors

(76) Inventor: James P. Huza, Greenville, NC (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/055,964

(22) Filed: Feb. 11, 2005

Related U.S. Application Data
(60) Provisional application No. 60/544,187, filed on Feb. 12, 2004.

(51) Int. Cl. A61F 13/15; A61F 13/20

(52) U.S. Cl. 604/359

(57) ABSTRACT

An apparatus for adsorbing flatulence and genitalia odor to be worn close to the body of a user, having a pad with a first elongated end and a second enlarged end, the ends conforming to the shape of the body of the user, a first layer of activated carbon fiber fabric interior of the pad and a second layer of fiber fabric joined to the first layer of activated carbon fiber fabric at its perimeter. Preferably, the activated carbon fiber fabric is about 100% activated carbon which is regenerative by heat. The second layer of fiber fabric is preferably a permeable material. If desired, the pad can be attached to the underwear/undergarment of the user by, for example, two or more eyelets and self-adhesive tie down strips. Preferably, the pad is also flexible, lightweight, washable, reusable, and reversible. A method of manufacturing an apparatus for adsorbing flatulence and genitalia odors as well as a method for adsorbing such odors are also described.

### RELATED APPLICATIONS

This application claims the benefit of U.S. provisional patent application No. 60/544,187 filed on Feb. 12, 2004.

### FIELD OF THE INVENTION

The present invention relates to an apparatus for flatulence and genitalia odor, a method of making the apparatus and a method of adsorbing flatulence odor. More particularly, the invention relates to an apparatus comprising a pad having first and second ends which conform to a user's body further having a layer of activated carbon fiber fabric and a second layer of fiber fabric.

### BACKGROUND OF THE INVENTION

The average person produces two to three pints of gas daily, which exits the body from the anus in the form of flatus or from the mouth in the form of belching. On average, flatus outbursts for a normal person range from 10-20 occurrences per day with the odoriferous component consisting mainly of sulfide gases. However, sufferers of, for example, irritable bowel syndrome, diabetes, diverticulitis, multiple sclerosis, aging and other gastrointestinal ailments can produce far more gas with more odors and emit it much more frequently. To minimize the odoriferous effects of these flatus events a wearable and regenerative flatulence odor control filter pad, incorporating air diffusion and odor adsorbing technology, were developed to eliminate the inherent conflict between someone with odoriferous intestinal gas and the people within his or her breathing zone.

There are several devices that deal with the problems associated with intestinal discharges including devices that filter and/or capture intestinal exudations of various kinds. Examples of such devices include those described in U.S. Pat. No. 4,182,335 of Matrullo, U.S. Pat. No. 4,490,145 of Campbell, U.S. Pat. No. 4,480,417 of Yabrov, U.S. Pat. No. 5,360,421 of Revelle, U.S. Pat. No. 5,665,081 of Grosse, U.S. Pat. No. 5,728,080 of Suyama, U.S. Pat. No. 5,957,907 of Oakley Sauer, and U.S. Pat. No. 6,313,371 of Conant, et al.

U.S. Pat. No. 4,182,335 of Matrullo describes an anal filter for protecting underwear from soiling, is attached to a person's anus solely be non-adhesive adherence to the anus and adjacent portions of the buttocks, due to a facing of fluffy fibrous material, and when attached, serves as a filter by being pervious to the discharge of gas through the anus but substantially impervious to complete passage of excreta carried by the gas or remaining on the anus from a bowel movement. A layer may be adhesively secured to the fibrous material and which prior to use of the anal filter can be pulled off from the fibrous material while pulling this material's fibers outwardly so as to give the material its fluffy characteristic.

U.S. Pat. No. 4,490,145 of Campbell describes an ostomy pouch having a filter element affixed to the outer pouch wall. The outer pouch wall has an aperture and the filter element includes a polymeric film cover and an insert of gas deodorizing material. The film cover also has an aperture and opposite ends of the insert overlie the two apertures.

Further, U.S. Pat. No. 4,880,417 of Yabrov describes an anal pad for preventing soiling of underwear due to undesired anal discharge. The anal pad includes a thin outer shell enclosing a semi-rigid shell. The semi-rigid shell may be a layer on one interior surface of the outer shell and may also partially cover the opposite interior surface of the outer shell. An absorbing layer is located within the outer shell and the semi-rigid layer. The absorbing layer contains glycerin which absorbs hydrogen sulfide gas and the combination of the absorbing layer and the semi-rigid shell acts as a sound muffling camera so as to decrease the noise due to flatus. The absorbing layer is made of a light porous material, the semi-rigid layer is made of compressed paper and the outer shell is made of a thin sheet of lignin paper. A layer of glue is provided on the outer surface of the anal pad for attaching it to a user's underwear.

Moreover, U.S. Pat. No. 5,360,421 of Revelle describes a washable and reusable absorbent garment designed to be fitted about the waist and legs and covering the groin and anal areas of a wearer. The garment includes a number of superimposed layers of textile material sewn together along their edges. Elastic material may be sewn in the material around the leg openings and waist to improve the fit. A liner material covers the inner surface of the garment. The liner layer of the garment or that portion which is next to the wearer's skin is a knitted bicomponent textile comprised of a brushed polyester outer layer which is interknitted with a nylon block co-polymer inner layer. The materials function to transfer moisture in both vapor and liquid form from the polyester side to the nylon side where it is dispersed and made available to the garment's second or absorbent layer. The two specific materials in the liner adjacent the wearer's skin and absorbent material work together to maintain a high degree of comfort for the wearer even after it has been wetted. The structural and functional integrity of the garment is maintained after repeated washings.

U.S. Pat. No. 5,665,081 of Grosse describes a pad for attenuating sound and absorbing odors from an anal region of an individual. The inventive device includes a pad member containing a volume of odor-absorbing granulated charcoal covered by a layer of filler material and encapsulated with a flexible web. A gluteus insert extends from the pad for positioning between the gluteus-maximus muscles and against the anal region of the user to attenuate noise emanating therefrom.

U.S. Pat. No. 5,728,080 of Suyama describes a degassing device for an artificial anal bag which is capable of storing the gas generated in the intestines, and of discharging the gas from the bag at a selected place where degassing causes no nuisance to others. It comprises an elongated tube having a filter loaded therein, an opening-and-closing plug attached to its head end and a diverging joint formed at its tail end. The diverging joint can be attached to the circumference of a joint opening, which is made in the upper part of the bag. When the bag is inflated with gas and excreta from the intestines, the patient can go to the men's room or somewhere for discharging the gas from the bag. Then, the excreta and liquid content are prevented from passing through the filter, only permitting the gas to flow therethrough. Thus, degassing can be put under the perfect control by the patient, so that there is no fear of permitting leakage of gas to cause nuisance to others or imprignation of his underwear with unpleasant odor before the patient is aware.

U.S. Pat. No. 5,957,907 of Sauer discloses an absorbent article that includes a close fitting bodyside liner having an opening therein. The opening defines a front end edge, a rear end edge, and a pair of laterally opposed side edges. The article also includes a pair of elastic members which extend longitudinally beyond at least one of the end edges of the opening in a laterally divergent direction. The elastic members may also extend at least partially along the side edges of the opening. The elastic members are configured to maintain the edges of the opening in contact with the wearer's buttocks and align the opening with the wearer's anal opening in use. The article may further include a liquid management material located on the bodyside liner in the front waist to absorb or contain liquid exudates.

Finally, U.S. Pat. No. 6,313,371 of Conant, et al. discloses a flatulence deodorizer which purports to be the first product to use a washable and reusable activated charcoal cloth as a deodorizer because of its effectiveness and efficiency in removing odor such that the thickness of the cloth can be significantly reduced without losing its effectiveness. The filter is worn taped to the inside of briefs or panties and, because of its slim profile, the wearer is comfortable and virtually unaware of its presence.

While these devices may be suitable for the purposes for which they were intended, there is a need for an improved apparatus or device which diffuses and adsorbs flatus gases as well as genitalia odors.

### SUMMARY OF THE INVENTION

Briefly stated, the present invention provides an apparatus including a pad incorporating air diffuser and odor adsorbing filters in the form of fabric to be worn by a user for adsorbing flatulence and/or genitalia odors. More specifically, the present invention provides an apparatus for adsorbing flatulence odors to be worn close to the body of a user, comprising a pad having a first elongated end and a second enlarged end, the ends conforming to the shape of the body of a user, a first layer of activated carbon fiber fabric interior of the pad and a second layer of fabric joined to the first layer of activated carbon fiber fabric at its perimeter. The activated carbon fiber fabric contains about 100% activated carbon, which can, preferably, be regenerated by heat. The activated carbon fabric is non-flammable at temperatures less than about 450-500° F. and the base material used has a better affinity to adsorb sulfide gases. The second layer of cloth is, preferably, a permeable material Further, if desired, the pad can be attached to the underwear of the user by, for example, two or more eyelets and self-adhesive tie down strips. Preferably, the pad is lightweight, flexible, washable, regenerative, reusable and reversible. A method of manufacturing an apparatus for adsorbing flatulence and/or genitalia odor to be worn close to the body of a user as well as a method of adsorbing flatulence and/or genitalia odor is further provided.

A primary object of the present invention is to provide an effective means of filtering intestinal gas odor. Another object of the present invention is to provide wearable means of adsorbing intestinal gas odor that will be least intrusive, lightweight, easy to use and comfortable for the wearer. Another object of the present invention is to provide a highly adsorptive apparatus providing a greater utilization of the activated carbon fiber filter area by the manufacturing methods employed. Another object of the present invention is to maximize the apparatus service life by minimizing peripheral material shredding during, for example, handling or washing, by the manufacturing methods employed. Still another object of the present invention is to provide a wearable means of controlling intestinal gas odor which can be regenerated and which is washable and reusable, yet inexpensive to purchase. Thus, the present invention is a safe, effective, cost efficient, lightweight, comfortable and a least intrusive apparatus, for adsorbing flatulence and/or genitalia odor in order to address the often-embarrassing problem of flatus outbursts. Additional objects of the present invention will appear as the description proceeds.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments, which are presently preferred. It should be understood, however, that the invention is not limited to the precise arrangements and instrumentalities shown.

In the drawings:

FIG. 1 is a perspective view, partially in cross-section, of an apparatus, including a pad, for adsorbing flatulence odor to be worn close to the body of a user in accordance with a first preferred embodiment of the present invention.

FIG. 2 is an enlarged cross-sectional view of a section of the apparatus of FIG. 1 taken along the line II-II of FIG. 1.

FIG. 3 is a perspective view, partially in cross-section, of an apparatus, including a pad, for adsorbing flatulence odor to be worn close to the body of a user showing the eyelets and self-adhesive tie down strips for attaching the apparatus in accordance with a second preferred embodiment of the present invention.

FIG. 4 is an enlarged cross-sectional view of a section of the apparatus of FIG. 3 taken along the line III-III of FIG. 3.

### DETAILED DESCRIPTION OF THE INVENTION

Certain terminology is used in the following description for convenience only and is not limiting. The words “right,” “left,” “lower,” and “upper” designate directions in the drawings to which reference is made. The terminology includes the words above specifically mentioned, derivatives thereof and words of similar import.

The present invention overcomes the shortcomings of the prior art by providing a versatile apparatus for adsorbing flatulence odor, which is worn close to the body of a user and is lightweight, flexible, washable, regenerative, reusable and reversible. The apparatus includes a pad having a first elongated end and a second enlarged end, the ends conforming to the shape of the body of a user; a first layer of activated carbon fiber fabric interior of the pad and a second layer of fabric joined to the first layer of activated carbon woven fiber fabric at its perimeter.

The activated carbon fiber fabric is an activated microporous carbonaceous adsorbent fiber filter produced in a flexible textile form, which adsorbs odors more effectively than other forms of activated carbon due to its microporous character. The activated carbon fiber fabric has a higher internal surface area, a greater tensile strength and a higher elastic rate than other carbon fabric used for controlling flatulence and, preferably, directly covers a surface of a user's body for optimal adsorption and minimal deflection of flatus outbursts. Because the activated carbon fiber fabric is more effective per unit area in removing flatus odor than other known fabric, the thickness of the fabric can be significantly reduced without losing effectiveness.

The methods of making the apparatus, applying the apparatus, and adsorbing flatulence odor are relatively simple and, unlike other apparatus for adsorbing flatulence odor, the present invention does not require the use of tape for adhering the apparatus to, for example, underwear of a user. Thus, the area and geometric shape of the pad are intended to be such that the pad can be sufficiently retained between, preferably, the user's body and underwear/undergarment without the need for a means for joining the apparatus to user's underwear/undergarment. It will, however, be recognized by those of ordinary skill in the art form this disclosure that, while various shapes and sizes of the pad can be used effectively, how effectively the pad is retained between, for example, the user's body and underwear/undergarment depends on the relationship of the size and shape of the pad to the size and shape of the user as well as the total thickness of the apparatus. Only one layer of fiber fabric is fastened to the activated carbon woven fiber fabric at its perimeter thus minimizing shedding after repeated washing while simultaneously maximizing adsorption. With this in mind and as described in more detail below, another embodiment of the present invention provides eyelets integral to a pad of the apparatus such that, for example, the apparatus can be joined, preferably using self-adhesive tie-down strips, to attach the apparatus to a user's underwear/undergarment.

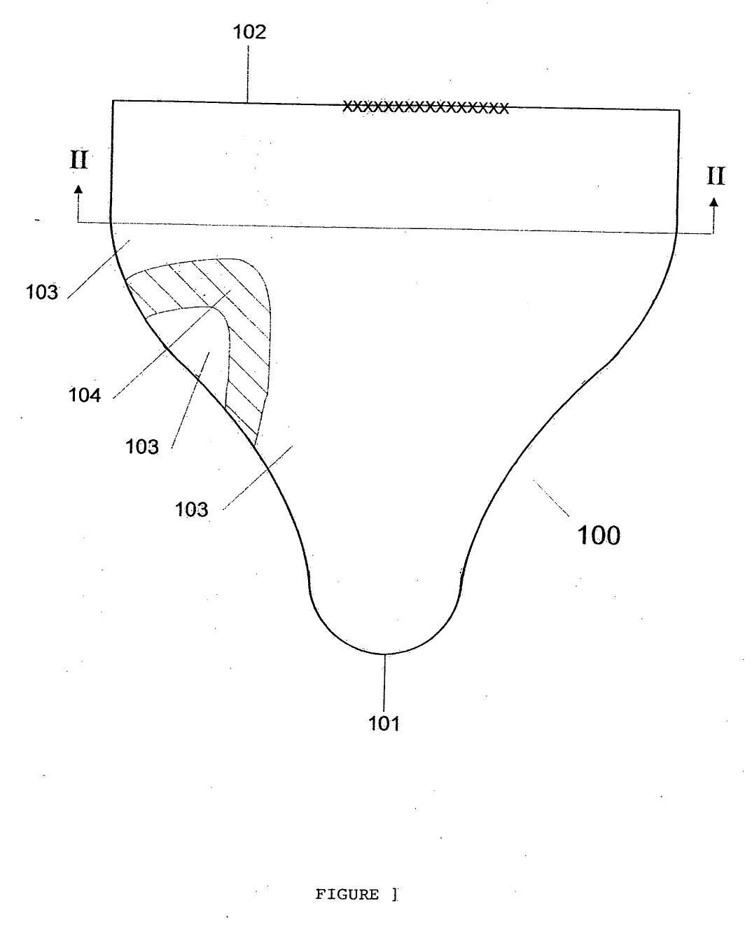

Referring to the drawings, wherein like reference numerals are used to indicate like elements throughout, there is shown in FIG. 1 a perspective view, partially in cross-section, of an apparatus for adsorbing flatulence odor to be worn close to the body of a user in accordance with a first preferred embodiment of the present invention. The apparatus includes a pad 100 having a first elongated end 101 and a second enlarged end 102, the ends conforming to the shape of the body of a user (not shown) wherein a first layer is preferable an activated carbon fiber fabric 103 which establishes the interior of the pad and a second layer of fiber fabric 104 is joined to the first layer of activated carbon fiber fabric 103 at its perimeter. The ends can be of various shapes and sizes although, preferably, the elongated end 101 is rounded to minimize chafing and the enlarged end 102 is approximately rectangular.

As also shown in FIG. 1, the pad 100 is constructed of an activated carbon fiber fabric 103 and, preferably, a single second layer of fiber fabric 104 such that the activated carbon fiber fabric 103 can be placed, preferably, directly in contact with a portion of the body of the user. The activated carbon fiber fabric 103 is, preferably, about 100% activated carbon and, most preferably, comprises preoxidized, polyacrylonitrile acrylic-based fibers and organic-based nitrides. It is also preferred that the activated carbon fiber fabric can be regenerated by applying heat to the activated carbon fiber fabric 103. For example, a commercially available clothes dryer or hair dryer can be used for about 5-10 minutes at 100-125° F., most preferably 7-10 minutes at 105-110° F. Preferably, the second layer of fiber fabric 104 is a permeable material and, most preferably, comprises at least one of polypropylene material and cotton material. When polypropylene is used in the second layer of fiber fabric 104, it is preferably a medical grade polypropylene material. It is also preferred that the pad 100 is scentless, although it will be recognized by those of ordinary skill in the art that the pad 100 can be scented, if desired, without departing from the spirit and scope of the invention.

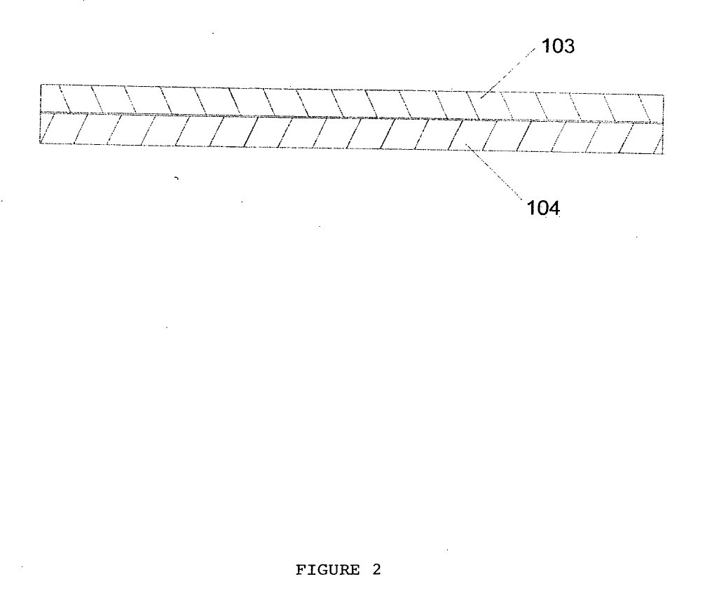

Referring now to FIG. 2, which is an enlarged cross-sectional view of a section of the apparatus of FIG. 1 taken along the line II-II of FIG. 1, the first layer of activated carbon fiber fabric 103 is joined to the second layer of fiber fabric 104 at, preferably, its perimeter. Several different means of joining the activated carbon fiber fabric 103 to the second layer of fiber fabric 104 can be used effectively. Preferably, the activated carbon fiber fabric 103 is peripherally joined to the second layer of fiber fabric 104 by at least one of stitching, gluing and heat sealing.

Stitching can be performed using, for example, cloth thread and a sewing-type machine or by hand. Several types of glues are known and available for joining fabric, including fabrics such as activated carbon fiber fabric. For example, Fabric-Tac™ is available from Beacon Chemical Co. Company, located in Mt. Vernon, N.Y. However, it will be recognized by those of ordinary skill in the art from this disclosure that other types of glues could be used, if desired, without departing from the spirit and scope of the invention. Heat sealing can be accomplished by applying heat to, for example, a glue known to be effective in joining fiber fabric materials. Various heat sealing means are known for joining fabrics which could be used, if desired, to join the activated carbon fiber fabric 103 to the second layer of fiber fabric 104 including, but not limited to, electronic sealing. It will be recognized by those of ordinary skill in the art from this disclosure that other types of joining means could be used, if desired, without departing from the spirit and scope of the invention.

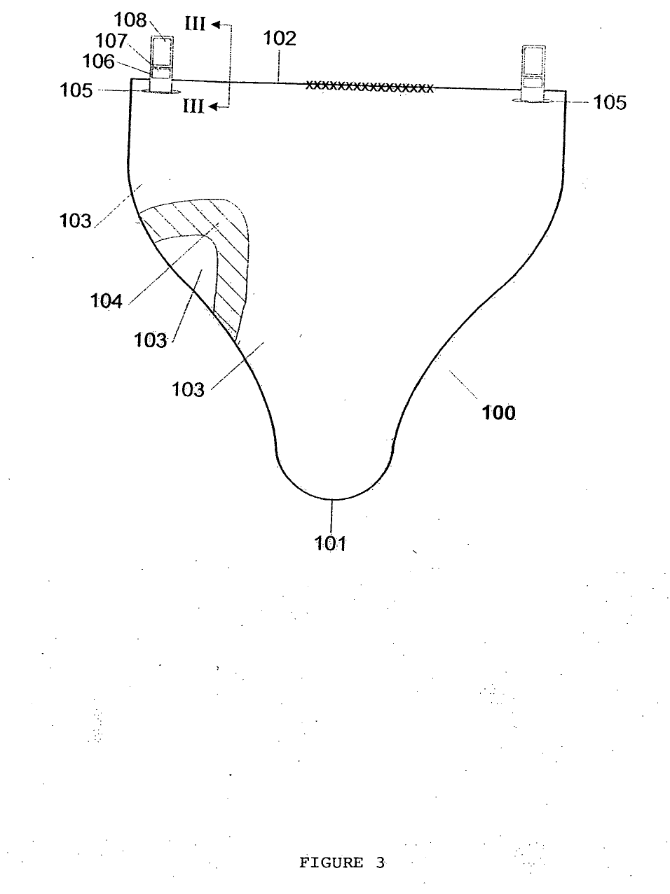

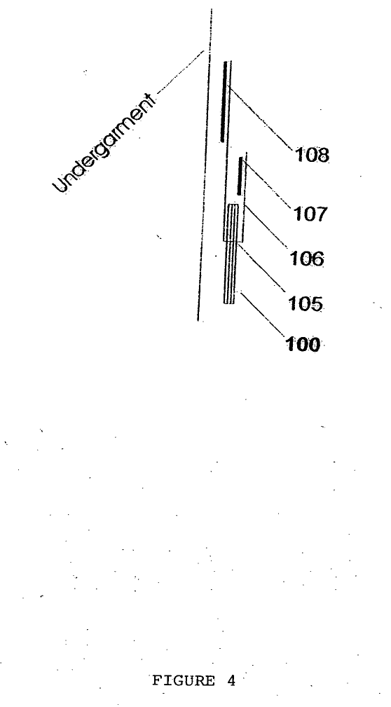

In another embodiment of the present invention, the apparatus further comprises a means for attaching the apparatus to the underwear of a user. FIG. 3 is a perspective view, partially in cross-section, of an apparatus for adsorbing flatulence odor to be worn close to the body of a user which shows eyelets 105 and self-adhesive tie down strips 106,107,108 for attaching the pad 100 in accordance with a second preferred embodiment of the present invention. Referring now to FIG. 4, which is an enlarged cross-sectional view of a section of the apparatus of FIG. 3 taken along line III-III of FIG. 3 which shows self-adhesive tie down strips 106,107,108 for attaching the pad 100 in accordance with a second preferred embodiment of the present invention. The second preferred embodiment shown in FIG. 3 is intended to only differ from the first preferred embodiment shown, for example, in FIG. 1 in that at least two eyelets 105 are available on the pad 100 for attaching the apparatus 100 to the underwear of the user (not shown) with the use of the self-adhesive tie down strips 106,107,108 preserving the integrity of the pad area after repeated use. It will be recognized by those of ordinary skill in the art from this disclosure that the at least two eyelets 105 could be used, if desired, in the second preferred embodiment as shown, for example, in FIG. 3 without departing from the spirit and scope of the invention. It will also be recognized by those of ordinary skill in the art from this disclosure that other types of attaching means could be used, if desired, including snaps, buttons or hook and loop fasteners, for example, without departing from the spirit and scope of the invention.

Another aspect of the present invention is to provide a method of manufacturing an apparatus for adsorbing flatulence and/or genitalia odor to be worn close to the body of a user which includes the steps of: joining a first layer of activated carbon fiber fabric to at least a second layer of fiber fabric at its perimeter to form a pad having a first elongated end and a second enlarged end; and conforming the pad to the shape of the body of a user. The method is intended to produce the apparatus previously described herein and therefore the features of the apparatus are incorporated herein by reference.

Still another aspect of the present invention is a method of adsorbing flatulence and/or genitalia odor using the apparatus already described such that when the pad 100, is properly placed against the body of a user, the pad 100, effectively and efficiently adsorbs flatulence odor. More specifically, the method comprises the steps of: joining a first layer of activated carbon fiber fabric to a second layer of fiber fabric at its perimeter to form a pad having a first elongated end and a second enlarged end; conforming the pad to the shape of the body of a user; and placing the pad against the body of the user.

It will be appreciated by those skilled in the art that changes could be made to the embodiments and methods described above without departing from the broad inventive concept thereof. It is understood, therefore, that this invention is not limited to the particular embodiments or methods disclosed, but it is intended to cover modifications within the spirit and scope of the present invention as defined by the appended claims.

1. An apparatus for adsorbing flatulence and genitalia odor to be worn close to the body of a user, comprising:
a) a pad having a first elongated end and a second enlarged end, the ends conforming to the shape of the body of a user;
b) a first layer of fiber fabric interior of the pad; and
c) a second layer of fiber fabric joined to the first layer of fiber fabric at about its perimeter.

2. The apparatus of claim 1, wherein the first layer comprises activated carbon.

3. The apparatus of claim 2, wherein the first layer is regenerative by heat.

4. The apparatus of claim 2, wherein the first layer is non-flammable.

5. The apparatus of claim 2, wherein the first layer comprises preoxidized, polyacrylonitrile acrylic-based fibers and organic-based nitrides.

6. The apparatus of claim 1, wherein the second layer comprises a permeable material.

7. The apparatus of claim 6, wherein the permeable material comprises at least one of polypropylene and cotton.

8. The apparatus of claim 1, wherein the first layer is joined to the second layer at about its perimeter by at least one of stitching material, glue and a heat sealing means.

9. The apparatus of claim 1, further comprising a means for attaching the pad to at least one of the apparatus and underwear of the user.

10. The apparatus of claim 9, wherein the means for attaching the pad comprises at least two eyelets and at least two self-adhesive tie down strips.

11. The apparatus of claim 1, wherein the pad is washable and reusable.

12. The apparatus of claim 1, wherein the pad is reversible.

13. A method of manufacturing an apparatus for adsorbing flatulence and genitalia odor to be worn close to the body of a user, comprising:
a) joining a first layer of activated carbon fiber fabric to a second layer of fiber fabric at its perimeter to form a pad having a first elongated end and a second enlarged end; and
b) conforming the pad to the shape of the body of a user.

14. A method of adsorbing flatulence and genitalia odor, comprising:
a) joining a first layer of activated carbon fiber fabric to a second layer of fiber fabric at its perimeter to form a pad having a first elongated end and a second enlarged end;
b) conforming the pad to the shape of the body of a user; and
c) placing the pad against the body of the user.
